(19) United States
(12) Patent Application Publication
(10) Pub. No.: US 2006/0280805 A1
(43) Pub. Date: Dec. 14, 2006

(54) Rouge free pharmaceutical water for injection (wfi) water system

(76) Inventor: Robert C. Livingston, Hyannis, MA (US)

Correspondence Address: GREENBERG TRAURIG, LLP, MET LIFE BUILDING, 200 PARK AVENUE, NEW YORK, NY, 10166, US

(21) Appl. No.: 10/595,669

(22) PCT Filed: Sep. 20, 2004
(86) PCT No.: PCT/US04/30624

§ 371 Date: May 3, 2006

Related U.S. Application Data
(60) Provisional application No. 60/504,484, filed on Sep. 19, 2003.

(51) Int. Cl. A61K 33/00; C02F 1/02

(52) U.S. Cl. 424/600; 210/600

(57) ABSTRACT

The present invention relates to rouge (corrosion) free pharmaceutical Water for Injection (WFI) systems. These systems are usually made of 316 stainless steel. Rouge is prevented by using a controlled atmosphere which does not contain carbon dioxide. Preferably, this controlled atmosphere is a mixture of about 80% nitrogen and about 20% oxygen.

This invention relates to a corrosion (rouge) free pharmaceutical Water for Injection (WFI) system.

### BACKGROUND

The high purity water industry began shortly after the widespread use of steam power utilized for manufacturing purposes during the industrial revolution. Softened water was soon identified as an urgent need by the not uncommon but devastatingly powerful explosions of steam boilers due to hardness scale. Filtration in conjunction with softening provided the standard pretreatment for the most common approach to the early deionization of water, distillation. Initially, distillation was the most reliable form of high purity water processing and remains a staple in the pharmaceutical industry to this day.

Modern ultra high purity water production began as a by-product of the nuclear age. The harnessing of nuclear energy demanded that ultra high purity water be available in large quantities and of exceptional purity to prevent the radioactive contamination of any contaminants of the heat transfer water and steam used in emerging nuclear technologies. Modern ion exchange and the production of ultra pure-18 MegOhm water was invented and perfected by Dr. Robert Kunin during the Manhattan Project. Reverse osmosis, a purification technique based on membrane technology, became commercially viable in the 1970's, and has become a central technology in high purity water processing.

Today, state of the art industrial high purity water systems utilize some or all of the following technologies to provide water that is approaching the theoretical limit for pure water.

Pretreatment
Sand
Carbon
Softener

Purification
Reverse Osmosis (RO) or Double Pass RO
CDI or EDI (Electrical Deionization)
Regenerable Mixed Bed Deionization

Post Treatment
Ultra-Violet TOC reduction Technology
Polishing Mixed Bed Deionization
Final Filtration or Ultra filtration (UF)

High purity water is utilized in numerous applications and in some way by virtually all technical research and manufacturing endeavors. A major component of modern semiconductor and biopharmaceutical manufacturing is a continuously available supply of exceedingly high purity water. Other major applications include the medical instruments, cosmetics, toiletries, photonics, aerospace, pharmaceutical and electronics manufacturing industries and power generation. Ultra high purity water cannot be bottled or stored, but must be manufactured as required, else it immediately degenerates into a lesser quality due to the “universal solvent” nature of deionized water. In order to manufacture water as required, users must utilize a high purity water system on site to provide ultra high purity water on demand. The systems range from small stills, through to wall mounted water systems to industrial manufacturing water systems housed in their own buildings to vast desalinization plants occupying acres and providing drinking water from seawater for entire communities. The high purity water is used by various industries for technical cleaning, degreasing, research, and as a stable and refined constituent of reagents, solutions and products.

Ample supplies of high purity water have become a required on-demand utility for modern technical manufacturing.

The pharmaceutical and related medical products industries are unique in that the US Federal government plays an important oversight role in their operation. The FDA (Food and Drug Administration) is responsible for protecting the nation's food and drug supply ensuring adequate safety in the manufacture, distribution and application of active pharmaceutical ingredients (API's). The FDA designates several qualities of water used in the manufacture of API's. The three grades of water in order of increasing quality are Process Water, Purified Water and WFI (water for Injection). WFI water is directed to be used in the final purification steps of active pharmaceutical ingredients (API's). WFI water is the only grade of water where the method of manufacture is specified, namely via distillation. WFI water is typically stored and distributed hot (65-80 C.) in 316 Stainless Steel piping. The combination of distilled water in contact with 316 Stainless Steel results in corrosion or rust which is called rouge in the pharmaceutical industry. This rouge contaminates the WFI water with rust particles and heavy metals in a profile that reflects the composition of the 316 Stainless Steel including iron, chrome, nickel, cobalt, tungsten and other trace metals. These contaminants are highly undesirable and until now have been tolerated in want of a solution to this vexing problem. The ubiquitous presence of rouge costs the pharmaceutical industry considerable expense in the cleaning and re-passivation of the stainless steel used in the water systems and associated piping. The downtime and chemical disposal fees associated with the cleaning and passivation of corroded systems is a tremendous expense contributing to the high cost of pharmaceuticals in the world.

### OBJECTS OF THE INVENTION

It is the purpose of this invention to eliminate rouge (rust and corrosion) and associated particulate contamination from pharmaceutical stainless steel WFI water systems.

This rouge-free WFI water system is unique in that:
The system employs state of the art technology in conjunction with a unique water processing distillation technique and storage and distribution system that eliminates the causes of rouge (corrosion) in WFI water systems.
It reliably provides unusually high quality, no microbiology product water exceeding the requirements for the following industry specifications:
   WFI Water
   USP Purified
   Reagent grade Type I water
   NACCLS Type I
The WFI product water quality is significantly improved via the reduction and even elimination of rust particles and heavy metal contaminants
The system is operated from any potable feed water supply.
The robust system design allows for extended use at high output capacity without the need for frequent or unscheduled maintenance or replenishment.

### DRAWINGS

FIG. 1 is a schematic of the present invention employing a multi-effect still.

FIG. 2 is a schematic of the present invention employing a vapor compression still.

### GENERAL DESCRIPTION OF THE INVENTION

Rouge is a phenomenon specific and endemic to the pharmaceutical industry despite the widespread use of high purity water in many other industries. The cause of rouge in pharmaceutical high purity water systems is the result of high purity water being distilled in contact with the atmosphere, and or allowed to equilibrate (degrade) with corrosive gases in the atmosphere, while in contact with 316 Stainless Steel. The mechanisms is as follows; high purity water readily adsorbs carbon dioxide which goes into solution as carbonic acid. The carbonic acid fosters a chemical reducing environment in the water that attacks the passivated (chromium oxide) surface of the stainless steel. The de-passivated surface permits iron to be exposed and oxidized resulting in rust (rouge). The aggressive high purity water readily attacks the iron in the de-passivated stainless steel and a variety of heavy metals dissolve into solution. The metal ions in solution will then react with oxygen and carbon dioxide in the WFI water creating various iron oxides and carbonates that comprise the colored corrosion deposits known as rouge marring the surfaces of the 316 Stainless Steel and contaminating the water. The rouging phenomenon accelerates and intensifies at elevated temperatures.

The present invention solves the problem of rouge as follows:

Pretreatment
High purity water system providing 18 MegOhm make-up water to still

Distillation
The WFI water is manufactured utilizing distillation as a final process step, as preferred and directed by the FDA and cGMP pharmaceutical practices. Vapor compression (VC) or Multi-effect (ME) distillation (stills) with controlled gaseous environment. This is accomplished with a nitrogen or inert gas. Residual oxygen is supplied by the water in most cases or must be provided with the inert gas or nitrogen as an approximately 10-20% oxygen residual, so long as no carbon dioxide is present.

Storage
Nitrogen or inert gas blanketed storage tank, preferably with about 10-20% oxygen, and no carbon dioxide.

Distribution
Distribution loop utilizing unique “closed loop” Arion Design for corrosion free circulation in 316 Stainless Steel piping.

This invention provides users with previously unobtainable high quality WFI water greater than 10 MegOhm resistivity, with less heavy metal contamination, continuously from any potable water source.

This water may be stored at elevated and/or ambient temperatures as is customary, but without the inevitable formation of corrosion products (rouge) and metal contamination of the WFI water, because the present invention prevents C0₂ from entering the water.

The water system will not need the periodic shutdown and disruption of service required to remove rouge and repassivate the 316 Stainless Steel surfaces of stills, storage tanks and stainless steel distribution loops and points of use.

### DETAILED DESCRIPTION OF THE INVENTION

This invention utilizes gas control during high purity water production, storage and distribution to prevent rouging (rusting) of the 316 Stainless Steel components used in Pharmaceutical High Purity Water production, specifically United States Pharmacopoeia (USP) grade WFI (Water for Injection) high purity water production.

WFI water systems are characterized by the following design characteristics:
1) Potable feed water source
2) Pretreatment conditioning of the water prior to Vapor Compression distillation
   a) filtration
   b) softening
   c) High pH reverse osmosis and/or membrane degasification for carbon dioxide control
   d) deionization—only if Multi Effect distillation is utilized
3) Distillation or some other acceptable means of deionizing and purifying water to WFI specifications
4) Storage of WFI water (ambient, cold or hot) without contamination (Carbon Dioxide) from the atmosphere
5) distribution of WFI water (ambient, cold or hot)

The Rouge free pharmaceutical water production process involves the prevention of carbon dioxide contamination of the high purity water during and after the purification process.

This is accomplished as follows:
Remove the carbon dioxide that is present in the feed water source.
Prevent the recontamination of the water with carbon dioxide during distillation or final purification.
Prevent the reintroduction of carbon dioxide during storage or distillation if in contact with 316 Stainless Steel.
   Mechanism

Carbon dioxide is typically present in feed water drinking sources, used as the source for the WFI water production. The carbon dioxide will not be present if the source water is to be completely deionized, as is the feed water to Multi Effect (ME) distillation process. The choices for pharmaceutical water distillation include:

| Distillation Process | Feed Water Requirement | CO2 Content |
|---|---|---|
| Single effect | Deionized (one MegOhm) | >0.01 ppm |
| Multi effect (ME) | Deionized (One MegOhm) | >0.01 ppm |
| Vapor Compression (VC) | Softened water | Present |

Step 1 Source Water Preconditioning

If a Multi Effect (ME) still is to be utilized, the still feed water should be deionized prior to distillation and will be free of carbon dioxide. The water so treated should have a resistivity of at least 15 MegOhms, preferably 18 MegOhm.

In the case of Vapor Compression (VC) distillation, softened water, free of chloramines and high levels of silica may be utilized for distillation. The water would need to be degassed and free of alkalinity and related forms of carbon dioxide, bicarbonate and carbonates. This is accomplished via softening, followed by a pH adjustment (4.0 or so) and degasification utilizing a membrane contactor style of degasification or reverse osmosis treatment operated at high pH (>8.5) followed by a membrane degasification contactor.

Step 2 Distillation

Industrial distillation occurs in contact with the atmosphere, which serves as the source for carbon dioxide contamination of the distilled product water. This is prevented by operating the stills in a controlled atmosphere such as nitrogen, argon, oxygen, or a nitrogen/oxygen mixture, so long as carbon dioxide from the atmosphere is not reintroduced into the distilled product water. This will be evident by the resistivity of the distilled product, which must exceed 10 MegOhm and should approach 18 MegOhm, an unheard of quality for distilled water.

Step 3 Storage

The distilled product will now go to a storage tank, blanketed with, for example, nitrogen, argon, oxygen, or a nitrogen/oxygen mixture to prevent the reintroduction of atmospheric carbon dioxide. Again, the resistivity of the water will be a true indicator of the absence of carbon dioxide, and it should exceed 10 MegOhms and preferably approach 18 MegOhms.

Step 4 Distribution

The water may be distributed at ambient or elevated (typically 65-80 C.) temperatures. The pharmaceutical industry prefers to utilize 316 Stainless Steel piping, which corrodes in the presence of high purity water containing carbon dioxide as carbonic acid in solution. The system will operate properly and will not rouge if steps one through three have been utilized. The rouge prevention process may be further enhanced if the water is circulated in the Arion “closed loop” approach and reheated once every 24 hours, as is also common.

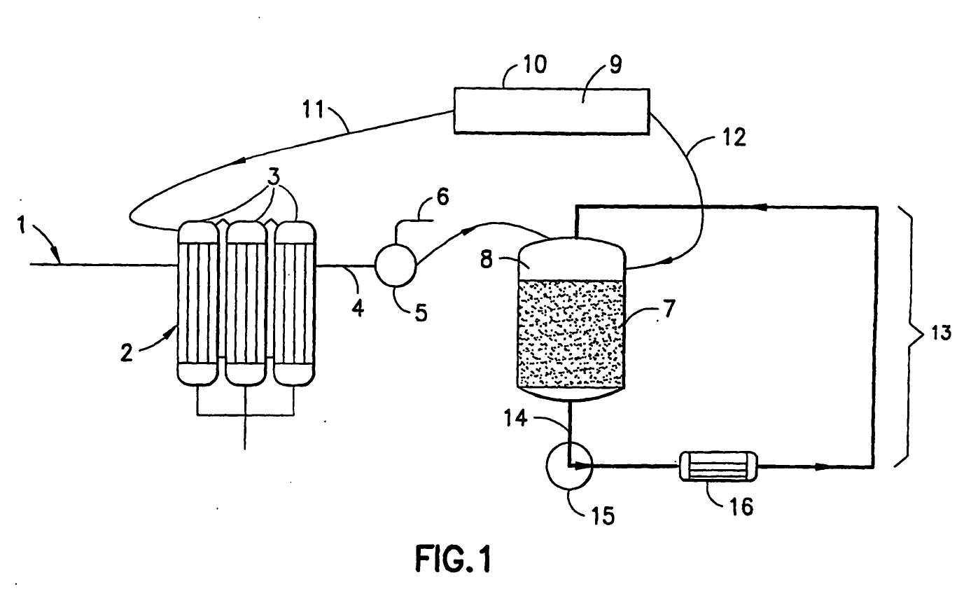

Referring now to the device of FIG. 1, carbon dioxide free water is introduced via a water feed (1) to a multi-effect still (2). This carbon dioxide free water preferably has a resistivity of at least about 18 MegOhms.

The carbon dioxide free water in the multi-effect still (2) must be maintained in a controlled atmosphere (3) which is substantially free of carbon dioxide. Preferably, this controlled atmosphere is an inert gas, nitrogen, oxygen, or a nitrogen/oxygen mixture. More preferably, this atmosphere is argon, nitrogen, oxygen, or a nitrogen/oxygen mixture. Even more preferably, the controlled atmosphere is a mixture of about 80% nitrogen and about 20% oxygen.

After the multi-effect still (2), the water is passed through a connection (4), and through a condenser (5) which contains a vent (6) to a WFI storage tank (7). In the WFI storage tank (7), the water is maintained in a controlled atmosphere (8) which is free of CO₂. In a preferred embodiment, the WFI storage tank's controlled atmosphere (8) is the same as the multi-effect still's controlled atmosphere (3). In one embodiment, this controlled atmosphere (designated 9) is stored in a container (10) with feeds (11 and 12, respectively) to the multi-effect still (2) and the WFI storage tank (7).

Optionally, the above system includes a unique, Arion Design close loop (13). In this close loop (13), the water in the WFI storage tank (7) is piped (14), preferably using 316 stainless steel piping, through a pump (15) and a heat exchanger (16) back into the WFI storage tank (7). In this way, the water in the WFI storage tank (7) can be maintained at a desired temperature. Of course, this close loop (13) maintains the water in a carbon dioxide free state. It is also understood that in this system, the WFI storage tank (7), condenser (5) connector (4), multi-effect still (2) and water feed (1) are all preferably constructed out of stainless steel, more preferably 316 stainless steel.

Using the above apparatus, WFI water can be made and stored in a rouge free state ready for use (exit feed not shown).

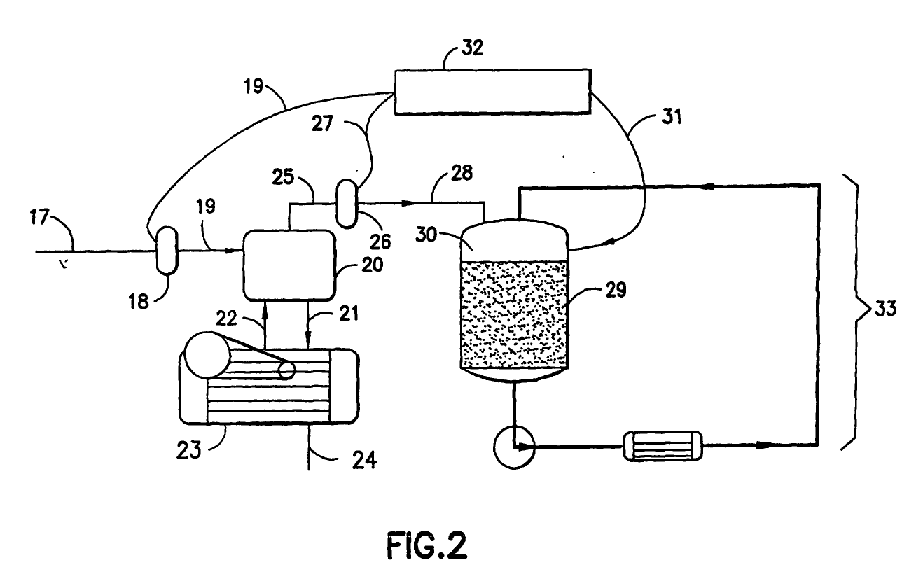

FIG. 2 shows a rouge free WFI water system using a vapor compression still. In this system, carbon dioxide free softened, reverse osmosis or deionized feed water is fed via water feed (17) to a first deaerator (18). This first deaerator (18) receives a first input (19) of a controlled atmosphere which is substantially free of carbon dioxide. Preferably, this controlled atmosphere is an inert gas, nitrogen, oxygen, or a nitrogen/oxygen mixture. More preferably, the controlled atmosphere is argon, nitrogen, oxygen, or a nitrogen/oxygen mixture. Even more preferably, the controlled atmosphere is a mixture of about 80% nitrogen and about 20% oxygen.

After the deaerator (18) the water is passed via a first connector (19) to a heat exchanger (20). Then, via a second connector (21) and a third connector (22), the water is passed to and from, respectively, a vapor compression still (23). The vapor compression still (23) optionally contains a drain (24).

Thereafter, the water is passed through a fourth connector (25) to a second deaerator (26). The second deaerator (26) contains a second input (27) of a controlled atmosphere which is substantially free of carbon dioxide. Preferably, this controlled atmosphere is an inert gas, nitrogen, oxygen, or a nitrogen/oxygen mixture. More preferably, the controlled atmosphere is argon, nitrogen, oxygen, or a nitrogen, oxygen mixture. Even more preferably, the controlled atmosphere is a mixture of about 80% nitrogen and about 20% oxygen. As shown in the embodiment of FIG. 2, the controlled atmosphere which is input into the second deaerator (26) is the same controlled atmosphere input into the first deaerator (18).

After the second deaerator (26) the water is transferred via a fifth connector (28) to a WFI storage tank (29). In the WFI storage tank (29) the water is stored under a blanket of controlled atmosphere (30) which is substantially free of carbon dioxide. The controlled atmosphere is introduced into the WFI storage tank (29) via a WFI storage tank connector (31). Preferably, the controlled atmosphere is an inert gas, nitrogen, oxygen, or a nitrogen/oxygen mixture. More preferably the controlled atmosphere is argon, nitrogen, oxygen, or a nitrogen/oxygen mixture. Even more preferably, the controlled atmosphere is a mixture of about 80% nitrogen and about 20% oxygen. As shown in FIG. 2, the controlled atmosphere of the blanket (30) in the WFI storage tank (29) is the same controlled atmosphere which is input into the second deaerator (26) and the first deaerator (18). In this embodiment, the controlled atmosphere can preferably be stored in a single container (32).

The system of FIG. 2 can also contain a unique, Arion design close loop (33). This close loop (33) is of similar configuration and function to the FIG. 1 close loop (13).

It is understood that the system of FIG. 2, like the system of FIG. 1, can preferably be constructed out of stainless steel, more preferably 316 stainless steel, especially the WFI storage tank (29).

Using the system of FIG. 2, WFI water can be made and stored in a rouge free state ready for use (exit feed not shown).

The above description and figures demonstrate various embodiments of the claimed invention. It is understood, however that the claimed invention is not limited to these embodiments only.

What is claimed is:

1. A method of preventing rouge in water which comprises:
a. providing a source of water which is substantially free of carbon dioxide;
b. distilling said water in an environment which is substantially free of carbon dioxide; and
c. storing said distilled water in an environment which is substantially free of carbon dioxide.

2. The method of claim 1 wherein the environment which is substantially free of carbon dioxide is an inert gas, nitrogen, oxygen or a mixture thereof.

3. The method of claim 1 wherein the distilling step and the storing step occur in the same environment which is substantially free of carbon dioxide.

4. A rouge free pharmaceutical water for injection purification system comprising:
a. a water intake;
b. a multi-effect still connected to said water intake, wherein said multi-effect still contains an internal controlled atmosphere which is substantially free of carbon dioxide;
c. a connector for the passage of water from the multi-effect still to a water for injection storage tank; and
d. a controlled atmosphere blanketing the water in the storage tank, wherein said controlled atmosphere is substantially free of carbon dioxide.

5. The rouge free water for injection purification system of claim 4, further comprising a closed loop with a heat exchanger attached to the storage tank, wherein the water is circulated through the heat exchanger to maintain the water at a desired temperature.

6. The rouge free water for injection purification system of claim 4 or 5, which is constructed out of stainless steel.

7. The rouge free water for injection purification system of claim 6, wherein the stainless steel is 316 stainless steel.

8. A rouge free pharmaceutical water for injection purification system comprising:
(a) a water intake;
(b) a first deaerator;
(c) a first input to said first deaerator, wherein said first input receives a supply of a carbon dioxide free controlled atmosphere for the first deaerator;
(d) a first connector connecting the first deaerator to a heat exchanger;
(e) a second connector connecting the heat exchanger to a vapor compression still;
(f) a third connector connecting the vapor compressions still to the heat exchanger;
(g) a fourth connector connecting the heat exchanger to a second deaerator;
(h) a second input into said second deaerator, wherein said second input receives a supply of a carbon dioxide free controlled atmosphere for said second deaerator;
(i) a fifth connector which connects the second deaerator to a water for injection storage tank; and
(j) a water for injection storage tank input, wherein said water for injection storage tank input receives a supply of carbon dioxide free controlled atmosphere for the water for injection storage tank.

9. The rouge free water for injection purification system of claim 8, further comprising a closed loop with a heat exchanger attached to the storage tank, wherein the water is circulated through the heat exchanger to maintain the water at a desired temperature.

10. The rouge free water for injection purification system of claim 8 or 9, which is constructed out of stainless steel.

11. The rouge free water for injection purification system of claim 10, wherein the stainless steel is 316 stainless steel.
